(19) United States
(12) Patent Application Publication
(10) Pub. No.: US 2021/0369607 A1
(43) Pub. Date: Dec. 2, 2021

(54) Antimicrobial Contact Lenses

(71) Applicant: Keramed, Inc., Fairfield, NJ (US)

(72) Inventor: Yichieh Shiuey, San Jose, CA (US)

(73) Assignee: Keramed, Inc., Fairfield, NJ (US)

(21) Appl. No.: 17/400,514

(22) Filed: Aug. 12, 2021

Related U.S. Application Data
(63) Continuation of application No. PCT/US19/17992, filed on Feb. 14, 2019.
Application No. PCT/US19/17992 is a continuation-in-part of application No. 16/954,322, filed on Jun. 16, 2020.
Application No. 16/954,322 is a continuation-in-part of application No. 16/387,465, filed on Apr. 17, 2019, now Pat. No. 10,792,245.
Application No. 16/387,465 is a continuation of application No. 15/260,251, filed on Sep. 8, 2016, now Pat. No. 10,307,369.
Application No. 15/260,251 is a continuation-in-part of application No. 17/001,953, filed on Aug. 25, 2020.
Application No. 17/001,953 is a continuation of application No. 16/387,465, filed on Apr. 17, 2019, now Pat. No. 10,792,245.
Application No. 16/387,465 is a continuation of application No. 15/260,251, filed on Sep. 8, 2016, now Pat. No. 10,307,369.

(51) Int. Cl. A61K 9/00; A61L 27/16; A61L 27/18; A61L 27/24; A61L 27/54; A61L 27/26; A61L 27/36; A61L 27/50; C08G 18/83; C08L 33/00

(52) U.S. Cl. CPC A61K 9/0051; A61L 27/16; A61L 27/18; A61L 27/24; A61L 27/54; A61L 27/26; A61L 2300/404; A61L 27/3687; A61L 27/50; C08G 18/83; C08L 33/00; A61L 2300/208; A61L 2430/16; A61L 27/3641

(57) ABSTRACT

An antimicrobial polymer for use in contact lenses includes at least one antimicrobial monomer; and at least one other monomer selected from an acrylic monomer, a hydrophobic acrylic monomer, a hydrophilic acrylic monomer, a vinyl monomer and/or a collagen monomer.

### FIELD OF THE INVENTION

The present invention relates generally to contact lenses and methods for their preparation and use, and more particularly, to antimicrobial polymers, obtained by copolymerizing at least one antimicrobial monomer with at least one other monomer for use in a contact lens.

### BACKGROUND OF THE INVENTION

Contact lenses are lenses that float on the human tear film. They are not physically embedded in the body, like an implant. A contact lens' purpose is to refract light to allow proper focus of light rays onto the retina and/or to provide a change to the cosmetic appearance of the eye.

Infections are serious complications of contact lenses. Bacteria can adhere to the contact lens and be transmitted to the ocular surface. An uncontrolled infection of the cornea due to contact lens use can result in loss of vision or even loss of the eye.

The most common strategy used in reducing the risk of infection is to use a contact lens storage solution with antimicrobial properties. Although this can be effective, it is reliant upon the patient to use the correct contact lens solution. For cost reasons, patients often will store contact lenses in saline, which has no antimicrobial properties. As such, there is a continual risk of infection.

Another strategy, which has been proposed in the past, is to infuse the polymer of the contact lens with an antibacterial metal ion. In particular, silver and copper metal have been proposed as agents to be infused into polymers for use in medical devices including contact lenses. Although, the use of free metal ions as an antibacterial agent within polymers has been used widely in commercial plastic goods and in some short-term disposable medical devices such as catheters, metal ions are known to be dangerous in the eye. Argyrosis is the medical term for silver toxicity of the eye. Argyrosis has been reported to result in a slate gray discoloration of the conjunctiva and iris. Argyrosis has also been found to result in cataracts and retinal maculopathy, both of which are vision threatening conditions. Copper toxicity in the eye results in a characteristic green ring around the cornea, which is termed a Kayser-Fleischer ring. Moreover, studies have demonstrated that copper toxicity can induce ocular complications such as intraocular inflammation (uveitis), hemorrhage, vitreous liquefaction, hypotony, iris ischemia and retinal damage. In addition, free metal ions may also leach out of the polymer over time and therefore, the polymer may lose its antimicrobial properties over time.

Dziabo and others have described the use of quaternary ammonium-containing organosilanes in the manufacture of an antimicrobial contact lens. However, the organosilanes pose a number of difficulties with regards to the manufacture of contact lenses. Quaternary ammonium-containing organosilanes may be used in the manufacture of silicone based contact lenses. However, manufacturing of silicone based contact lenses requires very expensive equipment and often needs to be done at very low temperatures. The expense of the equipment and special manufacturing environment makes the manufacture of silicone based contact lenses possible for only the largest companies. For most of the smaller manufacturers of contact lenses worldwide the polymer used in contact lenses is primarily methacrylate based. Other less commonly used contact lens materials include vinyl and collagen. Most organosilane compounds do not polymerize into a clear material in the presence of methacrylate, vinyl, or collagen and therefore cannot be used for the creation of anti-microbial contact lenses which are based on methacrylate, vinyl and/or collagen. Therefore, there is a need for an invention which allows methacrylate, vinyl and/or collagen based contact lenses to have antimicrobial properties.

For the stated reasons, there remains a need in the art for an improved composition and method of decreasing the risk of microbial infections related to contact lenses.

### SUMMARY OF THE INVENTION

The invention provides antimicrobial polymers for use in a contact lenses obtained by copolymerizing at least one antimicrobial monomer and at least one monomer selected from an acrylic, vinyl and/or collagen monomer. The invention also provides methods for preparing an antimicrobial polymer for use in a contact lenses, by reacting at least one antimicrobial monomer with at least one other monomer selected from an acrylic, vinyl and/or collagen monomer to provide the antimicrobial polymer, and using the antimicrobial polymer in the contact lenses.

The copolymers of the present invention are antimicrobial, biocompatible, and reversibly deformable and are also clear, translucent or opaque. In preferred aspects, the antimicrobial monomers of these co-polymers are not leachable after completion of the manufacture of the contact lens. Therefore, there will be no toxicity to the eye due to freely floating antimicrobial monomer. These characteristics are desirable for the optimal function of contact lenses. Moreover, because the entire co-polymer, not just the surface of the co-polymer, has antimicrobial properties, contact lenses made from these types of co-polymers will not lose their antimicrobial properties even if the surface of the implant becomes eroded over time. This is particularly important for contact lenses that are exposed to the surface of the eye, where blinking will cause erosion of the polymeric material. When the surface of the polymer of the present invention is eroded, the antimicrobial polymer beneath the surface will still kill microbes and thereby decrease the infection risk for the patient.

### BRIEF DESCRIPTION OF THE DRAWINGS

FIG. 1 illustrates an embodiment of the polymerization of an antimicrobial, clear, biocompatible, reversibly deformable polymer.

### DETAILED DESCRIPTION OF THE INVENTION

The following description is presented to enable a person of ordinary skill in the art to make and use embodiments described herein. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the disclosure. The word “exemplary” is used herein to mean “serving as an example illustration.” Any aspect or design described herein as “exemplary” is not necessarily to be construed as preferred or advantageous over other aspects or designs. Thus, the present disclosure is not intended to be limited to the examples described herein and shown but is to be accorded the scope consistent with the claims.

As used herein, reference to any biological drug includes any fragment, modification or variant of the biologic, including any pegylated form, glycosylated form, lipidated form, cyclized form or conjugated form of the biologic or such fragment, modification or variant or prodrug of any of the foregoing. As used herein, reference to any small molecule drug includes any salt, acid, base, hydrate, solvate, ester, isomer, or polymorph thereof or metabolite or prodrug of any of the foregoing. Abbreviations used herein have their conventional meaning within the chemical and biological arts.

It should be understood that the specific order or hierarchy of steps in the process disclosed herein is an example of exemplary approaches. Based upon design preferences, it is understood that the specific order or hierarchy of steps in the processes may be rearranged while remaining within the scope of the present disclosure. Any accompanying method claims present elements of the various steps in a sample order and are not meant to be limited to the specific order or hierarchy presented.

Accordingly, the present disclosure provides antimicrobial polymers for use in contact lenses, which are obtained by copolymerizing at least one antimicrobial monomer with at least one other monomer selected from an acrylic, vinyl and/or collagen monomer. The resulting antimicrobial polymer is antimicrobial, biocompatible, and which is also clear, translucent or opaque. These characteristics are desirable for the optimal function of contact lenses Moreover, the entire copolymer, not just the surface of the copolymer, has antimicrobial properties.

An important characteristic of the antimicrobial polymer is the un-leachability of the anti-microbial monomers and their immobilization within the polymer after completion of manufacturing. Anti-microbial monomers have been found to be toxic when they are exposed to animals in their free state. Small anti-microbial molecules are more likely to freely diffuse and may not be permanently fixed to the polymer after polymerization. For this reason, in preferred aspects the antimicrobial monomers of the present invention should contain at a minimum of 20 atoms in each molecule and preferably 40 or more atoms.

In one preferred aspect, the antimicrobial polymers of the present invention kill microorganisms on contact by causing their cells to burst. For example, the antimicrobial polymers of the present invention generally possess a positive charge, and can be readily adsorbed onto the negatively charged surface of the cell wall of the bacteria. Once adsorbed, the antimicrobial polymer chain then diffuses through the cell wall where it binds to and disrupts the cell membrane. The disruption of the cell membrane and subsequent leakage of cytoplasmic constituents leads to the death of the bacteria in a process known as bacteriolysis.

Most bacterial cell walls are negatively charged and therefore, most antimicrobial polymers are positively charged to facilitate the adsorption process. However, it is also possible to create a negatively charged antimicrobial polymer which would be suitable for killing positively charged bacterial cells.

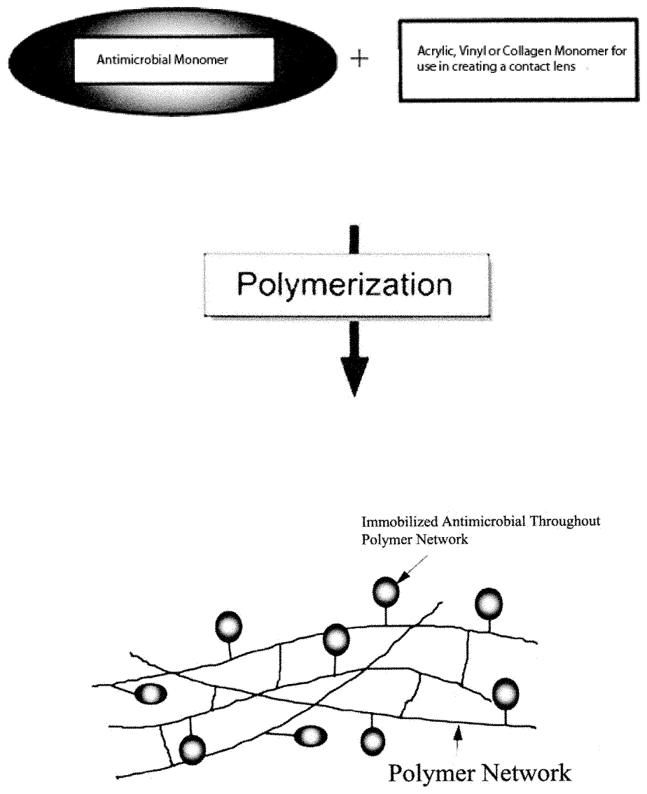

FIG. 1 illustrates an embodiment of the polymerization of an antimicrobial, clear, biocompatible, reversibly deformable polymer. In this figure, the antimicrobial polymers are obtained by copolymerizing at least one antimicrobial monomer with at least one monomer selected from an acrylic, vinyl and/or collagen monomer. After polymerization, the resulting polymeric network includes the immobilized antimicrobial polymer spaced throughout the network.

In an embodiment, the antibacterial monomer is selected from quaternary ammonium-salt based monomers which does not contain silicon. A non-limiting example of a quaternary ammonium salt based monomer is 1-[12-(methacryloyloxy)dodecyl]pyridinium bromide (MDPB):

CH₂═C(CH₃)C(O)O(CH₂)₁₂N⁺(C₅H₅) Br⁻MDPB

MDPB has been used as an antimicrobial monomer to reduce the risk of dental caries when copolymerized with dental adhesives and dental resins.

In other embodiments, the least one antimicrobial monomer is a quaternary ammonium salt-based monomer such as methacryloxylethyl cetyl dimethyl ammonium chloride (DMAE-CB).

CH₂═C(CH₃)C(O)O(CH₂)₂N⁺(CH₃)₂(CH₂)₁₅CH₃ Cl⁻DMAE-CB

It is also possible to increase the amount of antibacterial monomers that can be incorporated into polymeric materials and subsequently enhance the antibacterial activity by modifying the quaternary ammonium salt based monomers to have two polymerizable methacrylic moieties.

Thus, in other embodiments, the least one antimicrobial monomer is a quaternary ammonium salt based monomer such as 2-methacryloxyethyl dodecyl methyl ammonium bromide (MAE-DB):

CH₂═C(CH₃)C(O)O(CH₂)₂N⁺(CH₃)(CH₂)₂O(O)CC(CH₃)═CH₂(CH₂)₁₂CH₃ Br⁻MAE-DB

In other embodiments, the least one antimicrobial monomer is a quaternary ammonium salt based monomer such as 2-methacryloxyethyl hexadecyl methyl ammonium bromide (MAE-HB):

CH₂═C(CH₃)C(O)O(CH₂)₂N⁺(CH₃)(CH₂)₂O(O)CC(CH₃)═CH₂(CH₂)₁₆CH₃ Br⁻MAE-HB

In other embodiments, the least one antimicrobial monomer is a quaternary ammonium salt based monomer such as bis(2-methacryloxyethyl) dimethyl ammonium bromide (IDMA-1):

CH₂═C(CH₃)C(O)O(CH₂)₂N⁺(CH₃)₂(CH₂)₂O(O)CC(CH₃)═CH₂ Br⁻IDMA-1.

In other embodiments, the at least one antimicrobial monomer may differ based on the alkyl chain length. Examples of these include but are not limited to dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), dimethylamino docosyl methacrylate (DMADOM), and/or combinations thereof.

In other embodiments, the antimicrobial monomer may have a primary, secondary or tertiary amino group. Examples of these types of antibacterial monomers include but are not limited to ortho-, meta-, and/or para-dimethylaminomethyl styrene, N-[2-dimethylamino)ethyl]acrylamide, N-(2-aminoethyl)acrylamide, n-butylacrylamide, and diallyldimethyl ammonium salts.

In yet another embodiment, the cell growth inhibiting monomer is covalently linked to a cell growth inhibiting peptide. Examples of cell growth inhibiting peptides include: β-sheet peptides stabilized by two to four disulfide bridges (e.g., human α- and β-defensins, plectasin or protegrins), α-helical peptides (e.g., LL-37, cecropins or magainins), extended structures rich in glycine, proline, tryptophan, arginine or histidine (e.g., indolicidin), and loop peptides with one or disulfide bridge (e.g., bacteriocins).

In an embodiment, the at least one other monomer is selected from an acrylic, vinyl and/or collagen monomer. These monomers can undergo polymerization with the at least one antimicrobial monomer described above to provide the antimicrobial polymers for use in contact lenses.

In other embodiments, suitable acrylic monomers used to create a contact lens include at least one of the following monomers: glycerol monomethacrylate, 2-hydroxyethyl methacrylate, N-(2-hydroxypropyl)methacrylamide, hydroxypropyl methacrylate, poly(ethyleneglycol), monomethylether monomethacrylate, N-vinyl-2-pyrrolidone, isobutyl methacrylate, methyl methacrylate, N-octyl methacrylate, allyl phenyl ether, benzhydryl methacrylate, benzyl acrylate, N-benzyl methacrylamide, benzyl methacrylate, 2-(9H-carbazol-9-yl)ethyl methacrylate, 4-chlorophenyl acrylate, 1H, 1H,7H-dodecafluoroheptyl methacrylate, 1H, 1H,2H,2H-heptadecafluorodecyl acrylate, 1H, 1H,2H,2H-heptadecafluorodecyl methacrylate, 1H, 1H-heptafluorobutyl acrylate, 1H, 1H,3H-hexafluorobutyl acrylate, 1H, 1H,3H-hexafluorobutyl methacrylate, hexafluoroisopropyl methacrylate, 1H,1H,5H-octafluoropentyl acrylate, 1H, 1H, 5H-octafluoropentyl methacrylate, pentabromophenyl acrylate, pentabromophenyl methacrylate, pentafluorophenyl acrylate, pentafluorophenyl methacrylate, 1H, 1H, 3H-tetrafluoropropyl methacrylate, 2,4,6-tribromophenyl acrylate, 2,2,2-trifluoroethyl acrylate, 2,2,2-trifluoroethyl methacrylate, N-(3-aminopropyl)methacrylamide mono hydrochloride, 2-(N,N-dimethylamino)monoethyl methacrylate, methacrylic acid, 2-aminoethyl methacrylate hydrochloride, 4-(2-acryloxyethoxy)2-hydroxybenzophenone, phenyl acrylate, 4-methacryloxy-2-hydroxybenzophenone, 2-(2′ -methacryloxy-5′-methylphenyl)benzotriazole, 2-cinnamoyloxyethyl acrylate, cinnamyl methacrylate, glycidyl cinnamate, 2-naphthyl methacrylate, ethylene glycol dimethacrylate, 1,4-phenylene diacrylate, and poly(ethylene glycol) diacrylate.

In an embodiment, the at least one other monomer is selected a hydrophobic acrylic monomer. Examples of hydrophobic acrylic monomers include but are not limited to:

Monomers of phenylethyl acrylate, phenylethyl methacrylate and butanediol diacrylate, which form a copolymer of phenylethyl acrylate and phenylethyl methacrylate, cross linked with butanediol diacrylate (AcrySof® IQ) available from Alcon, A Novartis Division, 6201 South Freeway, Fort Worth, Tex. 76134-2001;

Monomers of ethyl acrylate, ethyl methacrylate, 2,2,2-trifluoroethyl methacrylate, cross linked with ethylene glycol dimethacrylate, which form a copolymer of ethyl acrylate, ethyl methacrylate, 2,2,2-trifluoroethyl methacrylate, cross linked with ethylene glycol dimethacrylate (Tecnis® (AMO)) available from Johnson & Johnson Vision Surgical, 1700 E St Andrew Pl, Santa Ana, Calif. 92705;

Monomers of phenylethyl methacrylate, n-butyl acrylate, and fluoroalkyl methacrylate, which form a cross linked copolymer of phenylethyl methacrylate, n-butyl acrylate, and fluoroalkyl methacrylate (AF-1® (HOYA)) available from Hoya Corporation, 7-5, Naka-Ochiai 2-chome, Shinjuku-ku Tokyo, Japan;

Monomers of phenylethyl acrylate, phenylethyl methacrylate, and butanediol diacrylate, which form a copolymer of phenylethyl acrylate and phenylethyl methacrylate, cross-linked with butanediol diacrylate (HI56) available from Contamac® Ltd., Carlton House, Shire Hill, Saffron Walden, Essex CB11 3AU;

Monomers of 2-phenylethyl acrylate and 2-phenylethyl methacrylate, which form a copolymer of 2-phenylethyl acrylate and 2-phenylethyl methacrylate (BENZ HF-1.2) available from Benz Research & Development Corporation, 6447 Parkland Drive, Sarasota, Fla. 34243; and

Monomers of 2-phenylethyl acrylate and 2-phenylethyl methacrylate, which form a copolymer of 2-phenylethyl acrylate and 2-phenylethyl methacrylate (Benz HF-2) available from Benz Research & Development Corporation, 6447 Parkland Drive, Sarasota, Fla. 34243.

In an embodiment, the at least one other monomer is selected a hydrophilic acrylic monomer. Examples of hydrophilic acrylic monomers include but are not limited to:

Monomers of hydroxyethyl methacrylate and methyl methacrylate, which form a copolymer of hydroxyethyl methacrylate and methyl methacrylate (CI26) available from Contamac® Ltd., Carlton House, Shire Hill, Saffron Walden, Essex CB11 3AU;

Monomers of hydroxyethyl methacrylate and methyl methacrylate, which form a copolymer of hydroxyethyl methacrylate and methyl methacrylate (MICS22) available from Contamac® Ltd., Carlton House, Shire Hill, Saffron Walden, Essex CB11 3AU;

Monomers of hydroxyethyl methacrylate and methyl methacrylate, which form a copolymer of hydroxyethyl methacrylate and methyl methacrylate (CI18) available from Contamac® Ltd., Carlton House, Shire Hill, Saffron Walden, Essex CB11 3AU;

Monomers of 2-hydroxyethyl methacrylate and 2-ethoxyethyl methacrylate, which form a copolymer of 2-hydroxyethyl methacrylate and 2-ethoxyethyl methacrylate (Benz IOL 125 available from Benz Research & Development Corporation, 6447 Parkland Drive, Sarasota, Fla. 34243; and

Monomers of 2-hydroxyethyl methacrylate and methyl methacrylate, which form a copolymer of 2-hydroxyethyl methacrylate and methyl methacrylate (BenzFlex 26) available from Benz Research & Development Corporation, 6447 Parkland Drive, Sarasota, Fla. 34243.

In other embodiments, the vinyl monomers used to create a contact lens include at least one of the following monomers: N-vinyl-2-pyrrolidone and/or N-vinyl carbazole monomers. Non-limiting examples of a vinyl monomer include but are not limited to vinyl esters (acrylates), vinyl carbonates (ROC(0)OCH═CH₂), and vinyl carbamates (R′R″NC(O)OCH═CH₂). Vinyl monomers can undergo polymerization with the at least one antimicrobial monomer described above to provide the antimicrobial polymers for use in contact lenses.

In other embodiments, the collagen monomers used to create a clear, opaque, or translucent, biocompatible contact lens include at least one of the following monomers: naturally derived type I-XXVIII collagen monomers, recombinant collagen monomers and fragments thereof, and/or synthetic collagen monomers and fragments thereof.

In an embodiment, the at least one other monomer is a collagen monomer. A single collagen molecule, tropocollagen, is used to make up larger collagen aggregates, such as fibrils. Fibrils are made up of three polypeptide strands, each of which has the confirmation of a left-handed helix. These three left-handed helices are twisted together into a right-handed triple helix or microfibril, a cooperative quaternary structure stabilized by hydrogen bonds. Each microfibril is then interdigitated with its neighboring microfibrils. Moreover, collagen monomers may be linked to one or more acrylate or vinyl monomers using various linkers. Collagen monomers can also undergo polymerization with the at least one antimicrobial monomer described above to provide the antimicrobial polymers for use in contact lenses. In an embodiment, the contact lenses may include collagen and N-isopropylacrylamide, collagen and 1-ethyl-3,3′(dimethyl-aminopropyl)-carbodiimide as well as collagen and N-hydroxysuccinimide (EDC/NHS).

While the inventive features have been particularly shown and described with reference to preferred embodiments thereof, it will be understood by those in the art that the foregoing and other changes may be made therein without departing from the sprit and the scope of the disclosure. Likewise, the various diagrams may depict an example architectural or other configuration for the disclosure, which is done to aid in understanding the features and functionality that can be included in the disclosure. The disclosure is not restricted to the illustrated example architectures or configurations but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described exemplary embodiments.

In the preceding description, the present disclosure is described with reference to specifically exemplary embodiments thereof. It will, however, be evident that various modifications and changes may be made thereto without departing from the broader spirit and scope of the present disclosure, as set forth in the claims. The specification and drawings are, accordingly, to be regarded as illustrative and not as restrictive. It is understood that the present disclosure is capable of using various other combinations and embodiments and is capable of any changes or modifications within the scope of the inventive concept as expressed herein.

What is claimed is:

1. An antimicrobial polymer for use in contact lenses, comprising:
at least one antimicrobial monomer; and
at least one other monomer selected from an acrylic monomer, a hydrophobic acrylic monomer, a hydrophilic acrylic monomer, a vinyl monomer and/or a collagen monomer.

2. The antimicrobial polymer according to claim 1, wherein the at least one antimicrobial monomer is a quaternary ammonium-based salt monomer.

3. The antimicrobial polymer according to claim 2, wherein the quaternary ammonium-based salt monomer is selected from 1-[12-(methacryloyloxy)dodecyl]pyridinium bromide (MDPB), methacryloxylethyl cetyl dimethyl ammonium chloride (DMAE-CB), 2-methacryloxyethyl dodecyl methyl ammonium bromide (MAE-DB), 2-methacryloxyethyl hexadecyl methyl ammonium bromide (MAE-HB), and/or bis(2-methacryloxyethyl) dimethyl ammonium bromide (IDMA-1).

4. The antimicrobial polymer according to claim 1, wherein the at least one antimicrobial monomer comprises a monomer having a primary, secondary or tertiary amino group.

5. The antimicrobial polymer according to claim 4, wherein the at least one antimicrobial monomer is selected from ortho-, meta-, and/or para-dimethylaminomethyl styrene, N[2-dimethylamino)ethyl]acrylamide, N-(2-aminoethyl)acrylamide, n-butylacrylamide, and/or a diallyldimethyl ammonium salt.

6. The antimicrobial polymer according to claim 1, wherein the at least one antimicrobial monomer is selected from dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and/or dimethylamino docosyl methacrylate (DMADOM).

7. The antimicrobial polymer according to claim 1, wherein the at least one antimicrobial monomer is covalently linked to an antimicrobial peptide.

8. The antimicrobial polymer according to claim 5, wherein the at least one antimicrobial monomer is selected from from β-sheet peptides stabilized by two to four disulfide bridges (e.g., human α- and β-defensins, plectasin or protegrins), α-helical peptides (e.g., LL-37, cecropins or magainins), extended structures rich in glycine, proline, tryptophan, arginine or histidine (e.g., indolicidin), and/or loop peptides with one or disulfide bridge (e.g., bacteriocins).

9. The antimicrobial polymer according to claim 1, wherein the acrylic monomer is selected from glycerol monomethacrylate, 2-hydroxyethyl methacrylate, N-(2-hydroxypropyl)-methacrylamide, hydroxypropyl methacrylate, poly(ethylene-glycol), monomethylether monomethacrylate, N-vinyl-2-pyrrolidone, isobutyl methacrylate, methyl methacrylate, N-octyl methacrylate, allyl phenyl ether, benzhydryl methacrylate, benzyl acrylate, N-benzyl methacrylamide, benzyl methacrylate, 2-(9H-carbazol-9-yl)ethyl methacrylate, 4-chlorophenyl acrylate, 1H, 1H,7H -dodecafluoroheptyl methacrylate, 1H,1H,2H,2H-heptadecafluorodecyl acrylate, 1H, 1H,2H,2H-heptadecafluorodecyl methacrylate, 1H, 1H-heptafluorobutyl acrylate, 1H, 1H,3H-hexafluorobutyl acrylate, 1H, 1H,3H-hexafluorobutyl methacrylate, hexafluoro-isopropyl methacrylate, 1H,1H,5H-octafluoropentyl acrylate, 1H,1H,5H-octafluoropentyl methacrylate, pentabromophenyl acrylate, pentabromophenyl methacrylate, pentafluorophenyl acrylate, pentafluorophenyl methacrylate, 1H,1H,3H-tetrafluoropropyl methacrylate, 2,4,6-tribromophenyl acrylate, 2,2,2-trifluoroethyl acrylate, 2,2,2-trifluoroethyl methacrylate, N-(3-aminopropyl)methacrylamide mono hydrochloride, 2-(N,N-dimethylamino)-monoethyl methacrylate, methacrylic acid, 2-aminoethyl methacrylate hydrochloride, 4-(2-acryloxyethoxy)2-hydroxybenzophenone, phenyl acrylate, 4-methacryloxy-2-hydroxybenzo-phenone, 2-(2′-methacryloxy-5′-methylphenyl)-benzo-triazole, 2-cinnamoyloxy-ethyl acrylate, cinnamyl meth-acrylate, glycidyl cinnamate, 2-naphthyl methacrylate, ethylene glycol dimethacrylate, and/or1,4-phenylene diacrylate, and poly(ethylene glycol) diacrylate.

10. The antimicrobial polymer according to claim 1, wherein the acrylic monomer is selected from methyl acrylate and methyl methacrylate.

11. The antimicrobial polymer according to claim 1, wherein the hydrophobic acrylic monomer is selected from:
monomers of phenylethyl acrylate, phenylethyl methacrylate and butanediol diacrylate, which form a copolymer of phenylethyl acrylate and phenylethyl methacrylate, cross linked with butanediol diacrylate,
monomers of ethyl acrylate, ethyl methacrylate, 2,2,2-trifluoroethyl methacrylate, cross linked with ethylene glycol dimethacrylate, which form a copolymer of ethyl acrylate, ethyl methacrylate, 2,2,2-trifluoroethyl methacrylate, cross linked with ethylene glycol dimethacrylate,
monomers of phenylethyl methacrylate, n-butyl acrylate, and fluoroalkyl methacrylate, which form a cross linked copolymer of phenylethyl methacrylate, n-butyl acrylate, and fluoroalkyl methacrylate,
monomers of phenylethyl acrylate, phenylethyl methacrylate, and butanediol diacrylate, which form a copolymer of phenylethyl acrylate and phenylethyl methacrylate, cross-linked with butanediol diacrylate,
monomers of 2-phenylethyl acrylate and 2-phenylethyl methacrylate, which form a copolymer of 2-phenylethyl acrylate and 2-phenylethyl methacrylate, and/or
monomers of 2-phenylethyl acrylate and 2-phenylethyl methacrylate, which form a copolymer of 2-phenylethyl acrylate and 2-phenylethyl methacrylate.

12. The antimicrobial polymer according to claim 1, wherein the hydrophilic acrylic monomer is selected from:
monomers of hydroxyethyl methacrylate and methyl methacrylate, which form a copolymer of hydroxyethyl methacrylate and methyl methacrylate,
monomers of hydroxyethyl methacrylate and methyl methacrylate, which form a copolymer of hydroxyethyl methacrylate and methyl methacrylate,
monomers of hydroxyethyl methacrylate and methyl methacrylate, which form a copolymer of hydroxyethyl methacrylate and methyl methacrylate,
monomers of 2-hydroxyethyl methacrylate and 2-ethoxyethyl methacrylate, which form a copolymer of 2-hydroxyethyl methacrylate and 2-ethoxyethyl methacrylate, and/or
monomers of 2-hydroxyethyl methacrylate and methyl methacrylate, which form a copolymer of 2-hydroxyethyl methacrylate and methyl methacrylate.

13. The antimicrobial polymer according to claim 1, wherein the vinyl monomer is selected from monomers of vinyl carbonate, vinyl carbamate, N-vinyl-2-pyrrolidone and/or N-vinyl carbazole.

14. The antimicrobial polymer according to claim 1, wherein the collagen monomer is selected from monomers of naturally derived type I-XXVIII collagen monomers, recombinant collagen monomers and fragments thereof, and/or synthetic collagen monomers and fragments thereof.

15. The antimicrobial polymer according to claim 1, wherein the antimicrobial polymer is clear.
